(19) United States
(12) Patent Application Publication
(10) Pub. No.: US 2006/0003372 A1
(43) Pub. Date: Jan. 5, 2006

(54) Integration of direct binding label-free biosensors with mass spectrometry for functional and structural characterization of molecules

(75) Inventors: Peter Li, Andover, MA (US); Bo Lin, Lexington, MA (US); Chris Williams, Franklin, MA (US); Lance Laing, Belmont, MA (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: SRU Biosystems, Inc., Woburn, MA (US)

(21) Appl. No.: 11/168,155

(22) Filed: Jun. 28, 2005

Related U.S. Application Data
(60) Provisional application No. 60/583,560, filed on Jun. 28, 2004.

(51) Int. Cl. C12Q 1/68; G01N 33/53

(52) U.S. Cl. 435/6; 435/7.1

(57) ABSTRACT

The invention provides methods for the detection, quantification, identification and structural analysis of one or more molecules. Mass spectrometry (MS) is not a universal detector as all molecules do not ionize equally well leading to poor signal to quantity information. MS can be optimized to identify the specific mass of a binding component when the presence of a material is known. Colorimetric resonant reflectance optical sensors provide a universal mass detector in that nearly all biological masses give equally proportional signals. The combined methods allow selection and or detection with quantification of all masses binding to the sensor with the ability to identify specific molecules by their individual masses and structure analyses.

### PRIORITY

This application claims the benefit of U.S. application Ser. No. 60/583,560, filed on Jun. 28, 2004, which is incorporate herein by reference in its entirety.

### BACKGROUND OF THE INVENTION

Detection assays combined with structural analysis can provide complementary information on function and structure of molecules. Previous work has demonstrated the utility of this combined approach for applications such as ligand fishing, epitope mapping, and amino acid sequencing, but only in the low throughput sample-limited context of a microfluidic channel-surface plasmon resonance (SPR)-based systems. See, Nelson et al., BIA/MS of Epitope-Tagged Peptides Directly from E. coli Lysate: Multiplex Detection and Protein Identification at Low-Femtomole to Subfemtomole Levels. Analytical Chemistry 1999, 71:2858-2865; Nelson et al., Biosensor chip mass spectrometry: A chip-based proteomics approach. Electrophoresis 2000, 21:1155-1163. Previously known methods of combining detection assays with structural analysis are extremely limited in utility by the subfemtomole quantities of bound material that can be recovered from a flow cell, and by the many sample injection/detection/elution cycles required to generate sufficient quantities of detectable material. See e.g., Williams & Addona, The integration of SPR sensors with mass spectrometry: possible applications for proteome analysis. Tibtech 2000, 18:45-48.

Methods are required in the art that enable these analytical techniques to be performed in a manner that is consistent with the throughput and cost goals of life science research.

### SUMMARY OF THE INVENTION

One embodiment of the invention provides a method of analyzing or identifying one or more molecules. The method comprises contacting a sample comprising the one or more molecules with a colorimetric resonant reflectance optical sensor such that one or more of the one or more molecules become immobilized to the colorimetric resonant reflectance optical sensor. The immobilized one or more molecules are eluted from the colorimetric resonant reflectance optical sensor and subjected to mass spectrometry analysis. The one or more molecules immobilized to the colorimetric resonant reflectance optical sensor can be detected. The one or more molecules immobilized to the colorimetric resonant reflectance optical sensor can be directly detected by a shift in peak wavelength value (PWV). The one or more molecules immobilized to the colorimetric resonant reflectance optical sensor can be detected using a label. A peak wavelength value (PWV) signal can also be detected. The detecting can comprise use of an indicator molecule of equal, greater, or lesser molecular mass of the one or more molecules immobilized to the colorimetric resonant reflectance optical sensor. The one or more molecules can be quantified. The one or more molecules can be immobilized to the colorimetric resonant reflectance optical sensor by one or more moieties on the surface of the colorimetric resonant reflectance optical sensor. The one or more moieties can be TiO, RaM Fc, avidin, biotin, an antibody, an antibody fragment, a nucleic acid molecule, protein A, hybrids of protein A, protein G, hybrids of protein G, protein L, hybrids of protein L, high density PVA, CHO or a combination thereof. The colorimetric resonant reflectance optical sensor can be coupled to a flow system for detection.

Another embodiment of the invention provides a method of analyzing or identifying one or more molecules. The method comprises contacting a sample comprising one or more molecules with a colorimetric resonant reflectance optical sensor such that one or more of the one or more molecules become immobilized to the colorimetric resonant reflectance optical sensor; obtaining any molecules that do not become immobilized to the colorimetric resonant reflectance optical sensor; and subjecting the any molecules that do not become immobilized to the colorimetric resonant reflectance optical sensor to mass spectrometry analysis.

Yet another embodiment of the invention provides a method of analyzing or identifying one or more molecules. The method comprises contacting a sample comprising one or more molecules with a flow based surface plasmon resonance sensor such that one or more of the one or more molecules become immobilized to the sensor; eluting the immobilized one or more molecules from the sensor; and subjecting the one or more molecules to mass spectrometry analysis. The one or more molecules immobilized to the sensor can be detected and/or quantified.

Still another embodiment of the invention provides a method of analyzing or identifying one or more molecules. The method comprises contacting a sample comprising one or more molecules with a surface plasmon resonant sensor such that one or more of the one or more molecules become immobilized to the sensor; obtaining any molecules that do not become immobilized to the sensor; subjecting the any molecules that do not become immobilized to the sensor to mass spectrometry analysis. The one or more molecules immobilized to the sensor can be detected and/or quantified.

### BRIEF DESCRIPTION OF THE DRAWINGS

FIG. 1 shows a sequence of steps for a colorimetric resonant reflectance optical sensor/MS analysis of a protein using an external MALDI-TOF plate. The colorimetric resonant reflectance optical sensor microplate is prepared with immobilized ligand that specifically detects a target protein from the test sample, registering a positive PWV shift. After binding, the bound target is eluted, mixed with MALDI matrix, and applied as a 1 μl spot to a standard MALDI plate for analysis.

FIG. 2 shows data from a colorimetric resonant reflectance optical sensor system showing the positive normalized PWV shift (ordinate values) for the attachment of antibody (hIgG & cIgG) and binding of antigen (Fab) and reduction of PWV shift as antigen (Fab) is eluted as a function of time (abscissa values) for the experiment. The elution volume with 10 mM glycine buffer pH 2 was ˜20 μL. About 1.2 nm PWV shift for the elution of Fab correlates with 3.6 ng/mm² from the ˜28 mm² surface area of the 6 mm diameter well of the 96-well microtiter colorimetric resonant reflectance optical sensor plate (i.e., about 100 ng of Fab are captured and eluted from the sensor surface). The Fab has an average molecular weight value of 22,300 Da.

FIG. 3A & FIG. 3B. FIG. 3A shows MALDI-TOF spectra for control of 2 pmol of Fab. FIG. 3B shows MALDI-TOF spectra for 1 μL of material eluted from a single well of the colorimetric resonant reflectance optical sensor in FIG. 2 spotted onto the MALDI surface with 1 μL sinapinic acid. The main peak for the expected material at 22,300 Da molecular weight is labeled. The two other significant peaks (11057 & 44967) are related to the major product peak.

FIG. 4 shows data from a colorimetric resonant reflectance optical sensor showing the positive normalized PWV shift (ordinate values) for the binding of antigen-A (Biogen Idec proprietary molecule and Ab) and reduction of PWV shift as antigen-A is eluted as a function of time (abscissa values) for the experiment. The elution volume with 10 mM glycine buffer pH 2 was ˜12 μL. About 80 pm PWV shift for the elution of antigen A correlates with 6.7 ng total mass from the ˜28 mm² surface area of the 6 mm diameter well of the 96-well microtiter colorimetric resonant reflectance optical sensor plate (i.e., about 0.3 pmol of the 17,900 Da molecule or 0.56 μg/mL or 28 nM). The experiment points out the sensitivity of the colorimetric resonant reflectance optical sensor system to detect small amounts of material binding specifically to the sensor.

FIG. 5 shows ESI-MS data for 6 μL of eluted material from the colorimetric resonant reflectance optical sensor shown in FIG. 4. The only significant peaks are related to the primary 17,900 expected product.

FIG. 6A shows a cross-sectional view of a sensor wherein light is shown as illuminating the bottom of the sensor; however, light can illuminate the sensor from either the top or the bottom. n_{substrate} represents substrate material. n₁ represents the refractive index of an optional cover layer. n₂ represents the refractive index of a grating. FIG. 6B shows another view of a sensor.

FIG. 7 shows an embodiment of a colorimetric resonant reflectance optical sensor comprising a one-dimensional grating.

FIG. 8 shows a resonant reflection structure consisting of a set of concentric rings.

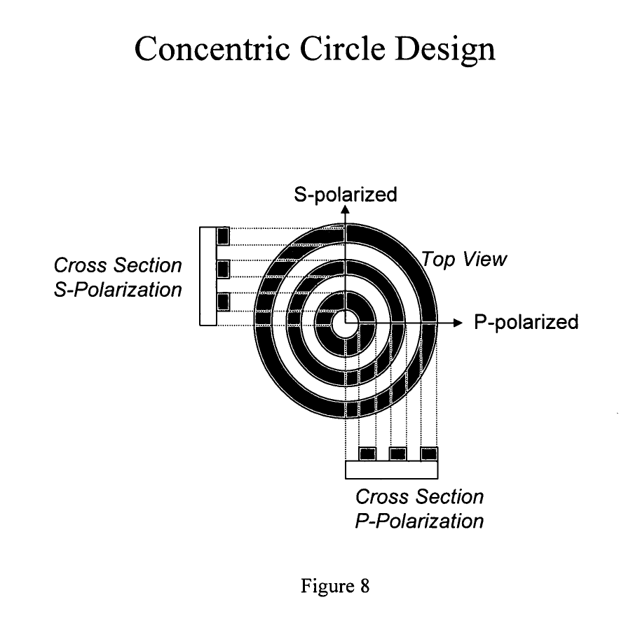

FIG. 9 shows a resonant reflective structure comprising a hexagonal grid of holes (or a hexagonal grid of posts) that closely approximates the concentric circle structure of FIG. 8 without requiring the illumination beam to be centered upon any particular location of the grid.

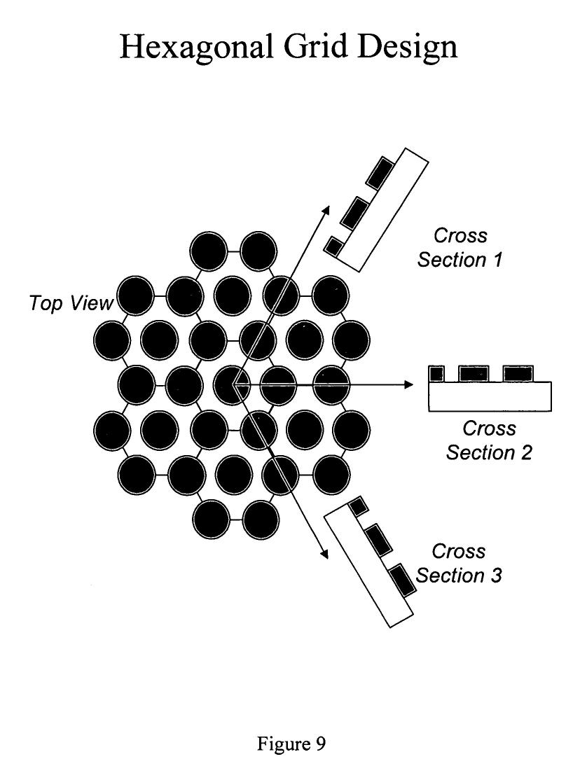

### DETAILED DESCRIPTION OF THE INVENTION

Within the past 30 years, a handful of technological developments in biochemical analysis methods and instrumentation have revolutionized conventional approaches to protein characterization, resulting in a progression of analytical instruments capable of providing information with higher sensitivity and accuracy. Two analytical approaches have found increasing use in protein characterization: direct binding assays and mass spectrometry (MS). Direct binding assays, such as those supported by colorimetric resonant reflectance optical sensors, are able to monitor biomolecular or cellular interactions as they occur between an immobilized receptor and a ligand in solution. The technology can determine the functional characteristics of proteins, and can be used to determine affinity parameters. MS-based techniques, such as matrix-assisted laser desorption/ionization time-of-flight (MALDI-TOF), and electrospray ionization (ESI), are used in the structural characterization of organic molecules, such as proteins, and inorganic molecules with analysis ranging from protein sequence verification through an accurate mass determination to protein identification via peptide mass mapping combined with database search.

Mass spectroscopy is not a universal detection method and often is not able to detect the presence of many materials if not properly tuned or focused on detecting them. With the present invention, the mass spectroscopist would know that material was indeed present and in what quantities and would thus be able to develop a better method to detect its presence, leading to the ability to identify the material by its mass and structure.

In one embodiment of the present invention, a method for the simplification of mass spectra can be achieved. Current trends in pushing mass spectroscopy technology to its greatest ends involve the mass analysis of very complex samples. The present invention allows for the deconvolution of the most complex samples by a sensor while preserving the mass analysis. A sensor allows for the specific selection and quantification of materials from complex media thus “cleaning” up a sample prior to its introduction to the mass analysis.

Mass spectra can often be crowded or have overlapping signals for larger molecular weight materials such as but not limited to biomaterials. This is especially true for complex samples from proteomics analyses, patient samples, pre-clinical samples from animals, whole cell lysates and the like. Removal of specific materials by the sensor (e.g., by immobilization of specific materials in a sample to the sensor and the use of the solution comprising the unbound specific material in MS analysis) can allow the mass spectra to become more easily determined for other masses. In an additional approach, the mass spectra can confirm the specific removal or selection of a material by the sensor. This could be accomplished by mass analysis of the material that did not adhere to the sensor.

One embodiment of the present invention has a significant benefit over previously described methods of combining a sensor with mass analysis. The present invention can be practiced with a static (meaning non-flow-based system) well based-sensor and as such offers significant advantage over other systems. The static based system is able to capture more transient interactions. Molecular interactions are described by rates for the amount of time that it takes for two molecules to form a pair and the amount of time that it takes for the two molecules to dissociate. When the dissociation rate is in a faster regime, the identification and analysis of the association is significantly challenging. A static system that is allowed to come to equilibrium has a far greater chance of capturing the interaction and preserving it for analysis than does any flow-based system described to date. As such the present invention, provides the mass analyst more sample to work with as a benefit to having any sample to work with at all.

Another embodiment of the invention provides a system where the colorimetric reflectance optical sensor is coupled to a flow system. The flow system can provide for high throughput screening of molecules.

Another embodiment of the current invention provides a method for the screening of individual or pools of small molecules. The world of small molecules for drug discovery is composed of large libraries of compounds many of which are thought to be pure or of substantially a single component while other parts of these libraries are composed of extracts from exotic organisms that have survived the rigors of environmental challenges. Many methods have been tried for the identification of specific binders of high affinity to human and veterinary disease targets. The present invention allows the detection and identification of members of the library that are binding with high affinity and specificity to said drug targets.

Another embodiment of the current invention provides a method for the screening of individual or pools of proteins. Following the great focus on human and pathogen genomics, the forefront of academic and pharmaceutical research has spent considerable time and effort studying proteomics or the presence, activity, and interaction of the vast number of proteins prescribed by the animal genome. The present invention provides methods for the study of the animal proteome by various techniques described herein.

Another embodiment of the invention provides a method for quantifying molecules. The molecules can be quantified by colorimetric resonant reflectance optical sensor, MS, or both colorimetric resonant reflectance optical sensor and MS. Molecules are quantified by colorimetric resonant reflectance optical sensor by immobilizing a ligand molecule to the colorimetric resonant reflectance optical sensor surface, and then detecting the PWV shift of the quantified molecule when it is exposed to the sensor+ligand surface. If the user had previously developed a calibration curve (PWV shift versus concentration of molecule), then the molecule concentration can be quantified by colorimetric resonant reflectance optical sensor. If the ligand is exposed to a complex test sample containing many analytes, then the PWV shift may be generated by a mixture of molecules. After the colorimetric resonant reflectance optical sensor detection, the bound molecules may be removed from the surface of the colorimetric resonant reflectance optical sensor and suspended in a solution. If MS analysis is performed on the solution, then the analytes that had been previously bound to the sensor can be quantified.

In another embodiment of the invention a binding constant is determined for one or more molecules. Samples comprising a range of analyte concentrations can be exposed to an immobilized ligand on the surface of a colorimetric resonant reflectance optical sensor. The binding constant for the ligand+analyte combination is determined. Where the sensor is exposed to a panel of different analytes at the same concentration, then the affinity of the analytes can be compared against each other by the magnitude of the sensor PWV signal. Additionally or alternatively, the bound material can be eluted from the sensor surface. The magnitude of the MS signal from the eluted solution can be measured to determine a binding constant.

Another embodiment of the invention provides method for preparing a Matrix Assisted Laser Desorption and Ionization/MS (MALDI/MS) matrix. The matrix can comprise colorimetric resonant reflectance optical sensor with one or more specific binding substances immobilized on the surface of the sensor. Optionally, the one or more specific binding substances can be bound to their respective binding partners. The one or more specific binding substances can be arranged in an array on the sensor surface.

Colorimetric Resonant Reflectance Optical Sensors

Colorimetric resonant reflectance optical sensors, which are direct binding sensors, have been described in detail in, for example, U.S. patent Ser. No. 09/929,957, filed Aug. 15, 2001; U.S. Pat. No. 930,353, filed Aug. 15, 2001; U.S. patent Ser. No. 10/415,037, filed Jan. 20, 2004; U.S. patent Ser. No. 10/399,940, filed Jan. 16, 2004; U.S. patent Ser. No. 09/930,352, filed Jan. 28, 2002; U.S. patent Ser. No. 10/058,626 filed Jan. 28, 2002; U.S. patent Ser. No. 10/201,878, filed Jul. 23, 2002; U.S. patent Ser. No. 10/196,059, filed Jul. 15, 2002; PCT US01/45455; PCT US03/01175; PCT US03/01298; Cunningham et al. Sensors and Actuators B, 81:316 (2002); Cunningham et al. Sensors and Actuators B, 85:219-226 (2002); Lin et al., Biosensors and Bioelectronics, 17:827 (2002); Cunningham et al., Sensors and Actuators B, 87:365 (2002), all of which are incorporated by reference herein in their entirety.

Colorimetric resonant reflectance optical sensors, alternatively referred to herein as sensors, can comprise a subwavelength structured surface (SWS). A SWS can create a sharp optical resonant reflection at a particular wavelength that can be used to track with high sensitivity the interaction of materials, including for example, specific binding substances or binding partners or both. A SWS acts as a surface binding platform for specific binding substances.

SWSs are an unconventional type of diffractive optic that can mimic the effect of thin-film coatings. (Peng & Morris, J. Opt. Soc. Am. A, Vol. 13, No. 5, p. 993, May 1996; Magnusson, & Wang, Appl. Phys. Lett., 61, No. 9, p. 1022, August, 1992; Peng & Morris, Optics Letters, Vol. 21, No. 8, p. 549, April, 1996). A SWS structure comprises a surface-relief grating, such as a one-dimensional, two-dimensional, or three dimensional grating in which the grating period is small compared to the wavelength of incident light.

The reflected or transmitted color of this structure can be modulated by the addition of molecules such as specific binding substances, binding partners, or both, or inorganic molecules to the upper surface of the cover layer or the grating surface. The dielectric susceptibility of the added molecules result in a modification of the wavelength at which maximum reflectance or transmittance will occur.

In one embodiment, a sensor, when illuminated with white light, is designed to reflect only a single wavelength or a narrow band of wavelengths. When specific binding substances or binding partners or both are attached or immobilized to the surface of the sensor, the reflected wavelength (color) is shifted. By linking specific binding substances to a sensor surface, complementary binding partner molecules can be detected without the use of any kind of fluorescent probe, or particle label or any other type of label. However, if desired one or more labels or indicator molecules can also be used. For example, a label molecule can be a dye, a fluorescent molecule, a bioluminescent molecule, and the like. An indicator molecule can be a biological or immuno-derived molecule of equal, greater, or lesser molecular mass of the one or more molecules immobilized to the colorimetric resonant reflectance optical sensor, e.g., a protein, peptide, nucleic acid, peptide nucleic acid, locked nucleic acid, and the like. The detection technique is capable of resolving changes of, for example, ˜0.1 nm thickness of protein binding, and can be performed with the sensor surface either immersed in fluid or dried.

A detection system consists of, for example, a light source that illuminates a small spot of a sensor at normal incidence through, for example, a fiber optic probe, and a spectrometer that collects the reflected light through, for example, a second fiber optic probe also at normal incidence. Because no physical contact occurs between the excitation/detection system and the sensor surface, no special coupling prisms are required and the sensor can be easily adapted to any commonly used assay platform including, for example, microtiter plates and microarray slides. A single spectrometer reading can be performed in several milliseconds, thus it is possible to quickly measure a large number of molecular interactions taking place in parallel upon a sensor surface, and to monitor reaction kinetics in real time.

This technology is useful in applications where large numbers of biomolecular interactions are measured in parallel, particularly when molecular labels would alter or inhibit the functionality of the molecules under study. High-throughput screening of pharmaceutical compound libraries with protein targets, and microarray screening of protein-protein interactions for proteomics are examples of applications that require the sensitivity and throughput afforded by the compositions and methods of the invention.

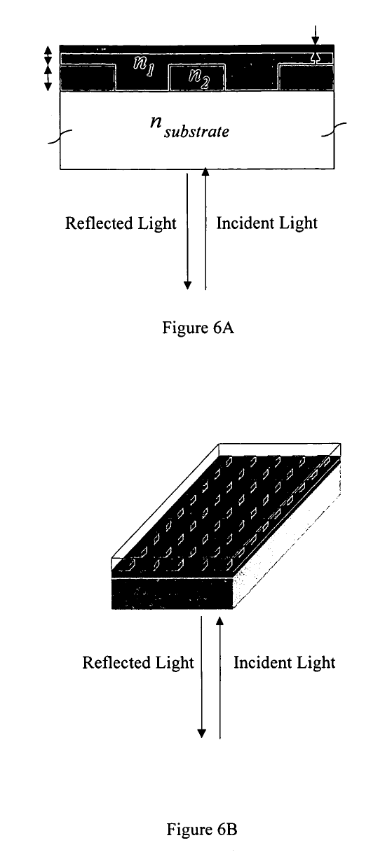

FIGS. 6A and 6B are diagrams of an example of a colorimetric resonant reflection optical sensor. In FIG. 6, n_{substrate} represents a substrate material. n₂ represents the refractive index of an optical grating. n₁ represents an optional cover layer. n_{bio} represents the refractive index of one or more specific binding substances. t₁ represents the thickness of the optional cover layer on the one-, two- or three-dimensional grating structure. t₂ represents the thickness of the grating. t_{bio} represents the thickness of the layer of one or more specific binding substances. In one embodiment, are n2>n1 (see FIG. 6A). Layer thicknesses (i.e. cover layer, one or more specific binding substances, or an optical grating) are selected to achieve resonant wavelength sensitivity to additional molecules on the top surface. The grating period is selected to achieve resonance at a desired wavelength.

A sensor comprises an optical grating comprised of a high refractive index material, a substrate layer that supports the grating, and one or more specific binding substances immobilized on the surface of the grating opposite of the substrate layer. Optionally, a cover layer covers the grating surface. An optical grating made according to the invention is coated with or comprises a high refractive index dielectric film which can be comprised of a material that includes, for example, zinc sulfide, titanium dioxide, tantalum oxide, and silicon nitride. A sensor of the invention can also comprise an optical grating comprised of, for example, plastic or epoxy, which is coated with a high refractive index material.

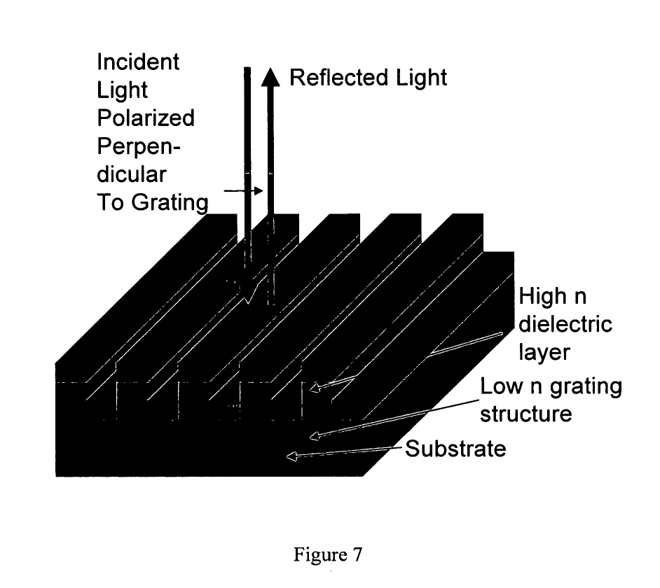

Linear gratings (i.e., one dimensional gratings) have resonant characteristics where the illuminating light polarization is oriented perpendicular to the grating period. A schematic diagram of one embodiment a linear grating structure with an optional cover layer is shown in FIG. 7. A colorimetric resonant reflectance optical sensor can also comprise, for example, a two-dimensional grating. A cross-sectional profile of a grating with optical features can comprise any periodically repeating function, for example, a “square-wave.” An optical grating can also comprise a repeating pattern of shapes selected from the group consisting of lines, squares, circles, ellipses, triangles, trapezoids, sinusoidal waves, ovals, rectangles, and hexagons. A linear grating has the same pitch (i.e. distance between regions of high and low refractive index), period, layer thicknesses, and material properties as a two-dimensional grating. However, light must be polarized perpendicular to the grating lines in order to be resonantly coupled into the optical structure in a manner that results in the most sharp resonant peak. Therefore, a polarizing filter oriented with its polarization axis perpendicular to the linear grating must be inserted between the illumination source and the sensor surface.

An optical grating can also comprise, for example, a “stepped” profile, in which high refractive index regions of a single, fixed height are embedded within a lower refractive index cover layer.

It is also possible to make a resonant sensor in which the high refractive index material is not stepped, but which varies with lateral position. For example, the high refractive index material of the two-dimensional grating, n₂, is sinusoidally varying in height. To produce a resonant reflection at a particular wavelength, the period of the sinusoid is identical to the period of an equivalent stepped structure. The resonant operation of the sinusoidally varying structure and its functionality as a sensor has been verified using GSOLVER (Grating Solver Development Company, Allen, Tex., USA) computer models.

A sensor of the invention can further comprise a cover layer on the surface of an optical grating opposite of a substrate layer. Where a cover layer is present, the one or more specific binding substances are immobilized on the surface of the cover layer opposite of the grating. Preferably, a cover layer comprises a material that has a lower refractive index than a material that comprises the grating. A cover layer can be comprised of, for example, glass (including spin-on glass (SOG)), epoxy, or plastic. Various polymers that meet the refractive index requirement of a sensor can be used for a cover layer. SOG can be used due to its favorable refractive index, ease of handling, and readiness of being activated with specific binding substances using the wealth of glass surface activation techniques. When the flatness of the sensor surface is not an issue for a particular system setup, a grating structure of SiN/glass can directly be used as the sensing surface, the activation of which can be done using the same means as on a glass surface.

Resonant reflection can also be obtained without a cover layer on an optical grating. For example, a sensor can comprise a substrate coated with a structured thin film layer of high refractive index material. Without the use of a planarizing cover layer, the surrounding medium (such as air or water) fills the grating. Therefore, specific binding substances are immobilized to the sensor on all surfaces of an optical grating exposed to the specific binding substances, rather than only on an upper surface.

Specific Binding Substances and Binding Partners

One or more specific binding substances are immobilized on the grating or cover layer, if present, by for example, physical adsorption or by chemical binding. A specific binding substance can be, for example, an organic or inorganic molecule, a nucleic acid, peptide, protein solutions, peptide solutions, single or double stranded DNA solutions, RNA solutions, RNA-DNA hybrid solutions, solutions containing compounds from a combinatorial chemical library, purified or mixtures of small molecule test compounds such as those used in the pharmaceutical industry to develop drug leads, individual or pools of proteins mixtures from various sources such as bacterial, viral, human, or other animal sources for proteome analyses, man-made, synthetic, or animal derived periplasmic extracts, antigen, polyclonal antibody, monoclonal antibody, single chain antibody (scFv), F(ab) fragment, F(ab′)₂ fragment, Fv fragment, purified or mixtures of immunobodies (e.g., scFv, sFab, F(ab), whole antibodies), small organic molecule, cell, virus, bacteria, polymer, TiO, RaM avidin, biotin, protein A, hybrids of protein A, protein G, hybrids of protein G, protein L, hybrids of protein L, high density PVA, CHO, or biological sample. A biological sample can be for example, blood, plasma, serum, gastrointestinal secretions, homogenates of tissues or tumors, synovial fluid, feces, saliva, sputum, cyst fluid, amniotic fluid, cerebrospinal fluid, peritoneal fluid, lung lavage fluid, semen, lymphatic fluid, tears, or prostatic fluid. A polymer can be selected from the group of long chain molecules with multiple active sites per molecule consisting of hydrogel, dextran, poly-amino acids and derivatives thereof, including poly-lysine (comprising poly-l-lysine and poly-d-lysine), poly-phe-lysine and poly-glu-lysine.

Preferably, one or more specific binding substances are arranged in an array of one or more distinct locations on a sensor. An array of specific binding substances comprises one or more specific binding substances on a surface of a sensor of the invention such that a surface contains many distinct locations, each with a different specific binding substance or with a different amount of a specific binding substance. For example, an array can comprise 1, 10, 100, 1,000, 10,000 or 100,000 distinct locations. Such a sensor surface is called an array because one or more specific binding substances are typically laid out in a regular grid pattern in x-y coordinates. However, an array can comprise one or more specific binding substance laid out in any type of regular or irregular pattern. For example, distinct locations can define an array of spots of one or more specific binding substances. An array spot can be about 50 to about 500 microns in diameter. An array spot can also be about 150 to about 200 microns in diameter. One or more specific binding substances can be bound to their specific binding partners.

An array on a sensor of the invention can be created by placing microdroplets of one or more specific binding substances onto, for example, an x-y grid of locations on a grating or cover layer surface. When the sensor is exposed to a test sample comprising one or more binding partners, the binding partners will be preferentially attracted to distinct locations on the microarray that comprise specific binding substances that have high affinity for the binding partners. Some of the distinct locations will gather binding partners onto their surface, while other locations will not.

A specific binding substance specifically binds to a binding partner that is added to the surface of a sensor of the invention. A specific binding substance specifically binds to its binding partner, but does not substantially bind other binding partners added to the surface of a sensor. For example, where the specific binding substance is an antibody and its binding partner is a particular antigen, the antibody specifically binds to the particular antigen, but does not substantially bind other antigens. A binding partner can be, for example, an inorganic molecule, an organic molecule, a nucleic acid, peptide, protein solutions, peptide solutions, single or double stranded DNA solutions, RNA solutions, RNA-DNA hybrid solutions, solutions containing compounds from a combinatorial chemical library, purified or mixtures of small molecule test compounds such as those used in the pharmaceutical industry to develop drug leads, individual or pools of proteins mixtures from various sources such as bacterial, viral, human, or other animal sources for proteome analyses, man-made, synthetic, or animal derived periplasmic extracts, antigen, polyclonal antibody, monoclonal antibody, single chain antibody (scFv), F(ab) fragment, F(ab′)₂ fragment, Fv fragment, purified or mixtures of immunobodies (e.g., scFv, sFab, F(ab), whole antibodies), small organic molecule, cell, virus, bacteria, polymer, TiO, RaM avidin, biotin, protein A, hybrids of protein A, protein G, hybrids of protein G, protein L, hybrids of protein L, high density PVA, CHO or biological sample. A biological sample can be, for example, blood, plasma, serum, gastrointestinal secretions, homogenates of tissues or tumors, synovial fluid, feces, saliva, sputum, cyst fluid, amniotic fluid, cerebrospinal fluid, peritoneal fluid, lung lavage fluid, semen, lymphatic fluid, tears, and prostatic fluid.

One example of an array of the invention is a nucleic acid array, in which each distinct location within the array contains a different nucleic acid molecule. In this embodiment, the spots within the nucleic acid microarray detect complementary chemical binding with an opposing strand of a nucleic acid in a test sample.

While microtiter plates are the most common format used for biochemical assays, microarrays are increasingly seen as a means for maximizing the number of biochemical interactions that can be measured at one time while minimizing the volume of precious reagents. By application of specific binding substances with a microarray spotter onto a sensor of the invention, specific binding substance densities of 10,000 specific binding substances/in² can be obtained. By focusing an illumination beam to interrogate a single microarray location, a sensor can be used as a microarray readout system. A sensor surface can be, for example, an internal surface of a microtiter plate.

Further, both the microarray and microtiter plate embodiments can be combined such that one or more specific binding substances are arranged in an array of one or more distinct locations on the sensor surface, said surface residing within one or more wells of the microtiter plate and comprising one or more surfaces of the microtiter plate, preferably the bottom surface. The array of specific binding substances comprises one or more specific binding substances on the sensor surface within a microtiter plate well such that a surface contains one or more distinct locations, each with a different specific binding substance or with a different amount of a specific binding substance. For example, an array can comprise 1, 10, 100, 1,000, 10,000 or 100,000 distinct locations. Thus, each well of the microtiter plate embodiment can have within it an array of one or more distinct locations separate from the other wells of the microtiter plate embodiment, which allows multiple different samples to be processed on one microtiter plate of the invention, one or more samples for each separate well. The array or arrays within any one well can be the same or different than the array or arrays found in any other microtiter wells of the same microtiter plate.

One or more specific binding substances can be immobilized to a sensor using methods well know in the art. Additionally, specific binding substances or binding partners of both can be eluted from the surface of a sensor using methods well known in the art.

Mass Spectrometry

Test samples can be analyzed by mass spectrometry using any type of mass spectrometer and any type of mass spectrometry methodologies. Additionally, mass spectrometers connected to other devices such as gas chromatographs, liquid chromatographs, super critical fluid chromatographs, and capillary electrophoresis devices can be used in the methods of the invention. Fourier transform ion cyclotron resonance (FT-ICR) spectrometer and time-of-flight (TOF) mass spectrometer, ElectroSpray Ionization Mass Spectrometry (ESI-MS), sector, and quadrupole devices can be used be used in the methods of the invention. Selected ion monitoring (SIM) can be used for quantitative analysis if desired. Mass spectrometry/mass spectrometry (MS/MS), isotope mass spectrometry, and elemental mass spectrometry techniques can also be used in the methods of the invention.

Methods of the Invention

Combination of colorimetric resonant reflectance optical sensor assays with mass spectrometry techniques, such as MALDI-TOF or ESI-MS, can be used to provide functional and structural characterization of organic and inorganic molecules, for example, proteins from serum. Additionally, the methods of the invention can be used in deorphaning of receptor analysis and protein identification by epitope tagging.

Taking advantage of the microplate-based format of colorimetric resonant reflectance optical sensors and the nondestructive nature of the label-free assay, a colorimetric resonant reflectance optical sensor/MS system is a multidimensional analytical approach that provides complementary information on, for example, protein function and structure, for example, post-translational alterations in a protein, in a simple, high throughput, highly sensitive platform. During colorimetric resonant reflectance optical sensor/MS analysis, the binding affinities of an immobilized ligand for analytes in, for example, 384 test samples are monitored simultaneously in real time. The analyte selectively retained or selectively not retained on the colorimetric resonant reflectance optical sensor microplate wells can be eluted (for the selectively retained molecules) and subsequently analyzed by mass spectrometry, such as MALDI-TOF or ESI-MS, which can confirm the identity of the affinity-retained analyte and detects multiple affinity-retrieved analytes. Used in this way, the sensor acts both as a sensitive instrument to quantify specific binding events to a target, and as a micropurification support for further analysis.

Where molecules that are selectively retained by the biosensor are analyzed only mass directly bound to the sensor surface is detected; dead cells and other precipitant materials that are chemically interacting with the sensor surface do not provide significant signal.

Due to the simplicity and low cost of the colorimetric resonant reflectance optical sensor reader instrument, colorimetric resonant reflectance optical sensor/MS integration is a cost effective approach for bringing new functionality to MS system users, and for differentiating MS systems from competing platforms.

There are many receptors of various predicted biological function that have no known ligand; such receptors are commonly referred to as orphans. “Ligand fishing” or “deorphaning” is the process by which these receptors are screened against a multitude of compounds or cell/tissue extracts to identify possible ligands for the receptor. See, Williams, Biotechnology match making: screening orphan ligands and receptors. Current Opinion in Biotechnology 2000, 11:42-46.

Similar techniques can also be applied to novel proteins that have no known binding partner. The most convenient methods for ligand fishing have traditionally been those based on direct binding, as these types of assays are not dependent upon the ligand's ability to activate a receptor or enzyme. Using a colorimetric resonant reflectance optical sensor/MS system, the orphan ligand is immobilized onto the bottom surface of all the wells in a colorimetric resonant reflectance optical sensor microplate. Each individual well is exposed to a separate test sample containing potential ligands for the orphan. Any well containing a high affinity binder for the orphan will register as a positive shift in peak wavelength value (PWV) in the colorimetric resonant reflectance optical sensor reader, registering a “hit.” Only the wells meeting the hit threshold are eluted for identification of the bound protein by MS.

Colorimetric resonant reflectance optical sensor/MS can be used with gene-tagging techniques for protein identification. See e.g., Nelson et al., Analytical Chemistry 1999, 71:2858-2865. First, a tag is fused into nominally unknown genes for the purpose of tracking proteins throughput expression and for selectively isolating protein from the expression system (such as E. coli). Using a highly selective tag-specific immobilized ligand on the bottom surface of the colorimetric resonant reflectance optical sensor microplate, the sensor is used to affinity isolate, detect, and quantify the tagged polypeptide retrieved from the expression system. Following colorimetric resonant reflectance optical sensor analysis, the masses of the tagged polypeptides are accurately determined by, for example, MALDI TOF analysis, which in turn are used for protein structural characterization such as sequence verification through a database search.

The colorimetric resonant reflectance optical sensor assay system can be easily interfaced with MS-based analysis systems to concurrently provide, for example, protein affinity and protein identification information. Deorphaning of targets and epitope mapping are other applications that would take advantage of this unique capability, given the sensitivity, throughput, and cost advantages inherent in the colorimetric resonant reflectance optical sensor system that have not previously been available. Preliminary experiments have been performed to confirm the basic approach for off-line colorimetric resonant reflectance optical sensor+MALDI-TOF analysis of a target Fab fragment. (See Examples). Due to the simplicity and low cost of colorimetric resonant reflectance optical sensor reader instruments, colorimetric resonant reflectance optical sensor/MS integration is a cost effective approach for bringing new functionality to MS system users, and for differentiating MS systems from competing platforms.

Another embodiment of the invention provides a method of analyzing or identifying one or more molecules comprising: contacting a sample comprising one or more molecules with a flow based surface plasmon resonance sensor such that one or more of the one or more molecules become immobilized to the sensor. The immobilized molecules can be eluted from the sensor or the non-immobilized molecules can be collected. These molecules are then subjected to mass spectrometry analysis. The one or more molecules immobilized to the sensor can be detected. The one or more molecules can be quantified.

All patents, patent applications, and other scientific or technical writings referred to anywhere herein are incorporated by reference in their entirety. The methods and compositions described herein as presently representative of preferred embodiments are exemplary and are not intended as limitations on the scope of the invention. Changes therein and other uses will be evident to those skilled in the art, and are encompassed within the spirit of the invention. The invention illustratively described herein suitably can be practiced in the absence of any element or elements, limitation or limitations that are not specifically disclosed herein. Thus, for example, in each instance herein any of the terms “comprising”, “consisting essentially of”, and “consisting of” can be replaced with either of the other two terms. The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by embodiments and optional features, modification and variation of the concepts herein disclosed are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

### EXAMPLES

### Example 1

Tandem BIND/MALDI-MS Experiment

Experiments were performed to show that material bound to a colorimetric resonant reflectance optical sensor surface can be efficiently eluted and analyzed by mass spectrometry. A capture antibody was adsorbed to a colorimetric resonant reflectance optical sensor surface. The corresponding antigen was allowed to bind to the antibody during application of the sample on the colorimetric resonant reflectance optical sensor. Any material bound specifically to the antibody was subsequently eluted from the surface. An aliquot of the eluted material was then applied for mass spectrometry analysis. The eluted material was mixed with the appropriate MALDI matrix and added to a MALDI plate and used for (TOF) MS analyses.

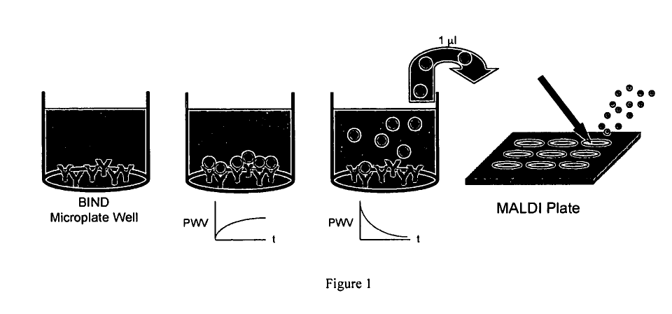

FIG. 1 shows the protocol used to combine the colorimetric resonant reflectance optical sensor technique with a MALDI type mass spectroscopy experiment. The process comprises adsorb antibodies on a colorimetric resonant reflectance optical sensor plate (0.1 mg/ml) and adding Anti-human IgG. The control is chicken IgY. IgG binds to the added Human Fab (MW average=22,300 Da) and a colorimetric resonant reflectance optical sensor signal is detected. The antigen eluted with glycine (10 mM, pH=2.0). The solution is removed from the colorimetric resonant reflectance optical sensor plate. A ZipTip® pipette tip is used to desalt the solution that is removed to facilitate MS analysis. One ul of the desalted material is added to a MALDI plate and mixed with 1 ul sinapinic acid. MALDI-MS data is obtained on Biogen Idec's ABI/Voyager system.

### Example 2

Matrix Assisted Laser Desorption Ionization (MALDI) Mass Spectroscopy (MS)

A colorimetric resonant reflectance optical sensor TiO sensor was pre-rinsed with PBS 3 times and left at room temperature for 30 min. A baseline reading was taken for a few min and 10 ul of 1 mg/ml of human IgG or chicken IgY was diluted into 90 ul PBS already in the well for a final concentration of the antibodies of 100 ug/mL. The protein was put into the well and allowed to bind to the TiO surface for 90 min. Unbound protein solution was removed from the well and the wells were rinsed 3 times each with 200 ul of PBS.

Another baseline reading was taken for a few minutes and then 1 ul of 1 mg/ml of anti-human IgG (Fab)2 was put into the wells, which were coated with either hIgG (Red) or cIgY (Yellow) and allowed to incubate for 60 min. All the unbound protein solution was removed from the wells and the wells were rinsed 3 times with PBS. The binding signal was monitored for stability for few min. Any retained Fab, bound by hIgG, was eluted with 30 ul of 10 mM Glycine pH 2 buffer in each well. The elution process was monitored on the sensor instrument and any eluted protein was collected for mass spectroscopic analyses.

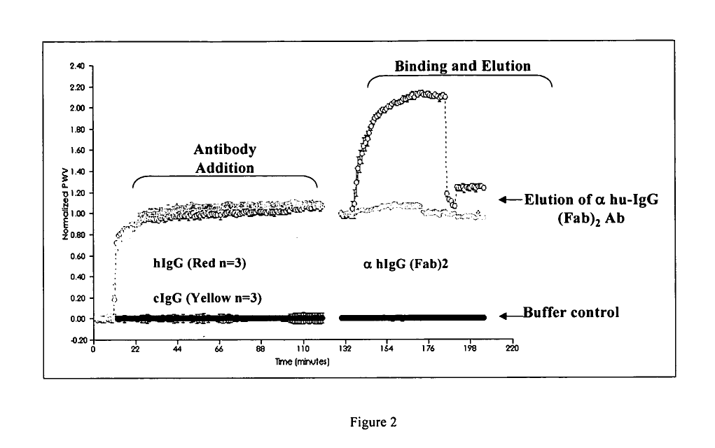

Specific interaction of anti-human IgG (Fab)2 and human IgG was detected using BIND. There was an undetectable signal for anti-human IgG (Fab)2 on chicken IgY (Yellow). See FIG. 2. About 0.8 nm ΔPWV of protein (=3 ng×0.8 nm×28 or 67 ng of protein) in 12 uL was eluted from the hIgG coated sensor surface for mass spectroscopy analyses.

### Example 3

Tandem BIND/MALDI-MS Experiment—MS Data

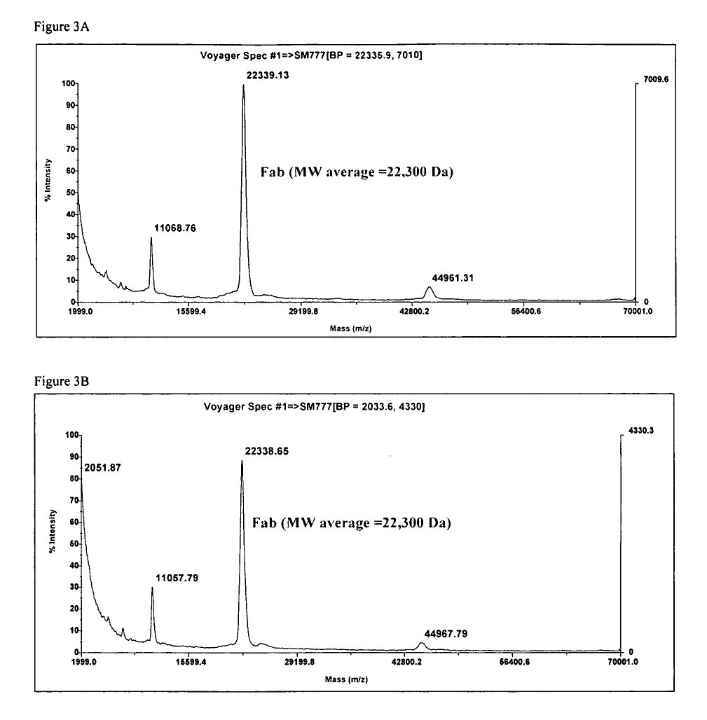

FIG. 3A shows the data from a control solution containing the Fab that was applied to the MS prior to exposure to the sensor surface. The primary peak is at 22300, the other two peaks are signature peaks related to the parent molecular mass

FIG. 3B shows the MALDI-MS data from the solution that was eluted from the sensor surface. The mass spectra is identical to the control spectra shown in FIG. 3A.

### Example 4

Tandem Colorimetric Resonant Reflectance Optical Sensor/ElectroSpray Ionization (ESI)—MS Experiment

Antibodies were adsorbed onto CHO colorimetric resonant reflectance optical sensor plate (low density aldehyde plate) (0.1 mg/mL). The antibody is a proprietary Human anti-Ag A antibody. Antigen A (20,000 Da) was added (˜20 mg/mL) to the antibody coated sensor and a BIND™ signal was detected. Unbound Ag A solution was removed from the sensor and the well was washed 3 times with PBS. Any Antigen A that was captured on the sensor was eluted from a single 6 mm diameter well with 12 uL glycine (10 mM, pH=2.0). The solution containing eluted protein was removed from the colorimetric resonant reflectance optical sensor plate. ESI-MS data was obtained on Biogen MS system.

The antigen protein was removed as evidenced by the differential signals determined by the sensor analysis. The MS data show definitive capture and mass determination of the specific antigen protein material that was eluted from the sensor. Antigen A binds at the 42 minute timepoint (abscissa values) to Antibody A that was immobilized onto CHO colorimetric resonant reflectance optical sensor. The unbound antigen A eluted mass/well (antigen)=>6.7 ng or 0.3 pmol. The eluted antigen concentration=>0.56 ug/mL or 28 nM.

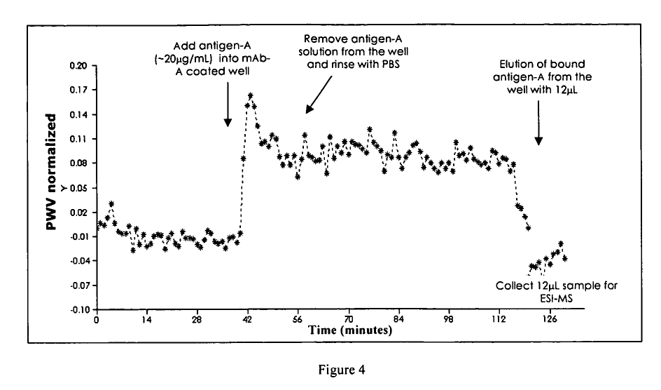

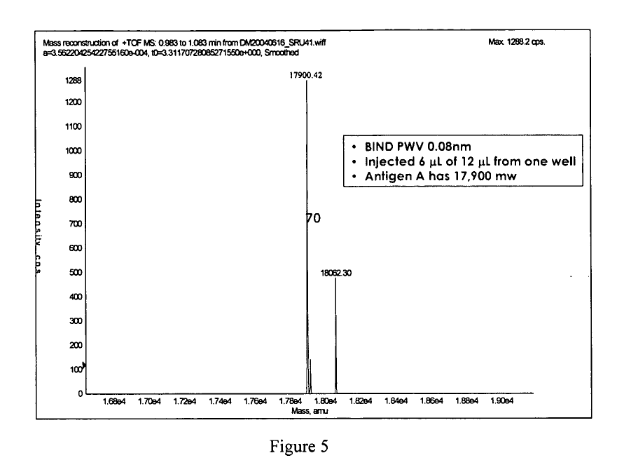

FIG. 4 shows the raw mass spectra from the ESI-MS experiment for the injection of 6 uL of the material eluted from a single 6 mm sensor well. FIG. 5 shows the mass analysis from the ESI-MS experiment for the injection of 6 uL of the material eluted from a single 6 mm sensor well. The parent peak is at the expected mass for the molecule of interest. The peak at 18052 mw is a signature peak related to the glycosylation of the primary peak. FIG. 5 demonstrates the ability of the sensor to “clean” up a sample thus providing a more simplified mass analysis experiment. The sensor was initially coated with protein A in order to capture a human monoclonal Ab in preclinical experiments. Following the addition of the mAb to the sensor surface, 100% human serum was added to the sensor. The data curves above begin with a zeroed reference point following the addition of the 100% human serum to the sensor surface. At 8 minutes in FIG. 5, either 1 uL or 5 uL of antigen in serum was added to different wells containing the mAb. Another control well contained a non-specific human IgG to which was added 5 uL of the antigen containing serum. The data clearly show that at 8.5 minutes the 1 & 5 uL mAb surfaces distinguish the different concentrations while the non-specific hIgG show no significant change. Following a wash of the sensor, the retained Ag is eluted and ready for mass analysis free from the other confounding materials from the 100% human serum. By the sensor response, we are able to see that we will be passing (0.18 nm×3 ng/mm2/nm×28 mm2) 15 ng of the Ag to the mass analyst.

The Examples demonstrate two extremes of the amount of materials a colorimetric resonant reflectance optical sensor is capable of providing for mass spectroscopic analyses. In addition, similar ESI-MS results have been collected using 50% serum samples on a colorimetric resonant reflectance optical sensor system.

We claim:

1. A method of analyzing or identifying one or more molecules comprising:
(a) contacting a sample comprising the one or more molecules with a colorimetric resonant reflectance optical sensor such that one or more of the one or more molecules become immobilized to the colorimetric resonant reflectance optical sensor;
(b) eluting the immobilized one or more molecules from the colorimetric resonant reflectance optical sensor; and
(c) subjecting the one or more molecules to mass spectrometry analysis, wherein one or more molecules are analyzed or identified.

2. The method of claim 1, wherein the one or more molecules immobilized to the colorimetric resonant reflectance optical sensor are detected.

3. The method of claim 1, wherein the one or more molecules are quantified.

4. The method of claim 1, wherein a binding constant is determined for the one or more molecules.

5. The method of claim 2, wherein a binding constant is determined for the one or more molecules.

6. The method of claim 2, wherein the one or more molecules immobilized to the colorimetric resonant reflectance optical sensor are directly detected by a shift in peak wavelength value (PWV).

7. The method of claim 2, wherein the one or more molecules immobilized to the colorimetric resonant reflectance optical sensor are detected using a label.

8. The method of claim 7, wherein a peak wavelength value (PWV) signal is also detected.

9. The method of claim 2, wherein detecting the one or more molecules immobilized to the colorimetric resonant reflectance optical sensor comprises the use of an indicator molecule of equal, greater, or lesser molecular mass of the one or more molecules immobilized to the colorimetric resonant reflectance optical sensor.

10. The method of claim 1, wherein the one or more molecules are immobilized to the colorimetric resonant reflectance optical sensor by one or more moieties on the surface of the colorimetric resonant reflectance optical sensor.

11. The method claim 10, wherein the one or more moieties comprise TiO, RaM Fc, avidin, biotin, an antibody, an antibody fragment, a nucleic acid molecule, protein A, hybrids of protein A, protein G, hybrids of protein G, protein L, hybrids of protein L, high density PVA, CHO or a combination thereof.

12. The method of claim 2, wherein the colorimetric resonant reflectance optical sensor is coupled to a flow system for detection.

13. A method of analyzing or identifying one or more molecules comprising:
(a) contacting a sample comprising one or more molecules with a colorimetric resonant reflectance optical sensor such that one or more of the one or more molecules become immobilized to the colorimetric resonant reflectance optical sensor;
(b) obtaining any molecules that do not become immobilized to the colorimetric resonant reflectance optical sensor; and
(c) subjecting the any molecules that do not become immobilized to the colorimetric resonant reflectance optical sensor to mass spectrometry analysis,
wherein one or more molecules are analyzed or identified.

14. The method of claim 13, wherein the one or more molecules are quantified.

15. The method of claim 13, wherein a binding constant is determined for the one or more molecules.
